(19) United States
(12) Patent Application Publication
(10) Pub. No.: US 2023/0233543 A1
(43) Pub. Date: Jul. 27, 2023

(54) COMPOUNDS FOR THE TREATMENT OF COVID-19

(71) Applicants: Dompe' Farmaceutici SPA, Milano (IT); FRAUNHOFER-GESELLSCHAFT ZUR FÖRDERUNG DER ANGEWANDTEN FORSCHUNG E.V., München (DE); KATHOLIEKE UNIVERSITEIT LEUVEN - KU LEUVEN R&D, Leuven (BE)

(72) Inventors: Andrea Rosario BECCARI, Napoli (IT); Candida MANELFI, Napoli (IT); Carmine TALARICO, Napoli (IT); Andrea ZALIANI, Hamburg (DE); Gerd GEISSLINGER, Frankfurt am Main (DE); Philip GRIBBON, Hamburg (DE); Pieter LEYSSEN, Landen (BE)

(21) Appl. No.: 17/923,420

(22) PCT Filed: May 6, 2021
(86) PCT No.: PCT/EP2021/061977

§ 371 Date: Nov. 4, 2022

(30) Foreign Application Priority Data
May 6, 2020 (EP) 20173318.5

(51) Int. Cl. A61K 31/4535; A61K 31/473; A61P 31/14

(52) U.S. Cl. CPC A61K 31/4535; A61K 31/473; A61P 31/14

(57) ABSTRACT

The present invention relates to compounds that are able to inhibt functional proteins of COVID-19 virus, SARS-Cov-2.

### FIELD OF THE INVENTION

The present invention relates to compounds that are able to inhibit functional proteins of COVID-19 virus, SARS-CoV-2.

### STATE OF THE ART

Coronaviruses (Covs) are a large family of single-stranded, enveloped RNA viruses that belong to the Coronaviridae family. Until recently, the limited number of coronaviruses known to be circulating in humans were considered as relatively harmless respiratory human pathogens, causing mild infections. However, in the last years the emergence of the severe acute respiratory syndrome coronavirus (SARS-CoV) and the Middle East Respiratory Syndrome (MERS) virus revealed that some coronaviruses are able to cause severe and sometimes fatal respiratory tract infections in humans (Pereira, H et al, Andrewes’ Viruses of Vertebrates 1989, 5th ed., 42-57; Holmes, K.V. et al., Virology 1996, 1: 1075-1093). The first known case of SARS-CoV occurred in Foshan, China, in November 2002 and new cases emerged in mainland China in February 2003. The first emergence of MERS-CoV occurred in June 2012 in Saudi Arabia. These events demonstrated that the threats of CoVs should not be underestimated and that it is of paramount importance to advance the knowledge on the replication of these viruses and their interactions with the hosts to develop treatments and vaccines.

In December 2019, atypical pneumonia cases caused by a novel coronavirus occurred in China. The World Health Organization (WHO) named the virus as SARS-CoV-2 and the related disease as COVID-19.

The virus spread rapidly worldwide, and on 11 Mar. 2020 the WHO declared SARS-CoV-2 infection as a pandemic. Most people infected with COVID-19 experience mild to moderate respiratory illness (fever, fatigue, dry cough and dyspnea) and recover without requiring special treatments. Older people and those with underlying medical problems like cardiovascular disease, diabetes, chronic respiratory disease, and cancer are more likely to develop serious illness.

Phylogenetic analysis of CoVs of different species indicated that SARS-CoV-2 could have originated from Chinese horseshoe bats, but the intermediate transmission vehicle has not yet been identified (Dong, N. et al. Microbiology 2020). According to this study, SARS-CoV-2 belongs to a novel type of bat coronavirus owing to a high degree of variation from the human SARS virus. SARS-CoV-2 is the seventh member of the family of CoVs that infects humans.

Analysis of SARS-CoV-2 genome sequences obtained from patients during the beginning of the outbreak demonstrated that they share 79.5% sequence identity to those of SARS-CoV. Like SARS-CoV, SARS-CoV-2 enters target cells through an endosomal pathway and using the same cell entry receptor, Angiotensin-Converting Enzyme II (ACE2). In particular, the spike protein at the surface of the virus binds to ACE2, through its receptor-binding domain (RBD), thereby enabling the entry of the virus into cells. After entry into the host cells, the viral RNA is released into the cytoplasm and is then translated in two polyproteins, pp1a and pp1b, that are cleaved into nonstructural proteins NSP1-16 by the viral proteases papain-like protease (PLpro) and 3C-like protease (3CL-Pro) (Moustaqil et al, Emerging Microbes & Infections 2021, 10(1), 178-195).

NSPs have important roles in replication and transcription (Rajarshi et al, Gene 2021, 768: 145313).

PLpro and 3CL-Pro are also responsible for cleaving mediators of host antiviral immune response, thus providing a mechanism by which the virus evades the immune system (Choudhury et al, In Silico Pharmacol 2021, 9: 26; Liu et al, Viruses 2020, 12: 1039).

An important structural protein for activity of Coronaviruses is the nucleocapsid protein (N-protein), which packs the RNA genome forming a helical ribonucleoprotein (RNP) complex interacting with the other structural proteins during virion assembly.

Therefore, the above SARS-CoV-2 proteins and the complex between Spike and ACE2 represent effective therapeutic targets in SARS-CoV-2 for preventing replication and proliferation of the virus.

The identification and development of new drugs is a lengthy process that is not thus adequate to face the emergency of the immediate global challenge of COVID-19 outbreak. Repurposing of drugs already tested as safe in man or approved for different therapeutic is a rapid response solution, since the pharmacokinetic, toxicological, and manufacturing data for the drigs are already available, thus allowing immediate application in clinical setting(Anand et al., Science 2003;300(5626):1763-7).

### SUMMARY OF THE INVENTION

The present inventors have carried out the analysis of a library containing commercialized drugs and clinical candidates safe in man or characterized up to late clinical stage and have selected by Computer Associated Drug Design a number of molecules that are able to bind and inhibit the activity of one or more proteins of SARS-Cov-2 selected from 3CL protease, PL protease, N-protein, NSP3, NSP6, NSP9, NSP12, NSP13, NSP14, NSP15, NSP16 or to inhibit the Spike-ACE2 interaction. The inventors have then confirmed the inhibitory activity of the selected molecules on SARS-Cov-2 by in vitro screening.

The results obtained support the use of the compounds identified in the treatment of infections from SARS-Cov-2.

In details, the compounds identified as having inhibitory activity on the virus are Proflavine, Raloxifen, Mequitazine, N-tert-Butylisoquine, Isofloxythepin, Succinobucol and Hypericin. All these compounds are well known approved drugs or drug candidates for different indications.

Proflavine is the generic name of the compound 3,6-diaminoacridine having the following general formula (I):

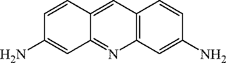

The compound is an antiseptic bacteriostatic against many gram-positive bacteria. It has been used in the form of the dihydrochloride and hemisulfate salts as a topical antiseptic, mainly in wound dressings, and was also formerly used as a urinary antiseptic. Raloxifen is the generic name of 1-[6-Hydroxy-2-(4-hydroxyphenyl)benzo[b]thien-3-yl]-1-[4-[2-(1-piperidinyl)ethoxy]phenyl]methanone, having the following general formula (II):

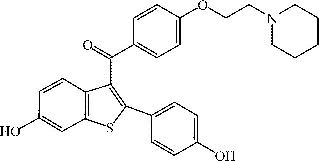

The compound is a selective benzothiophene estrogen receptor modulator (SERM) with lipid lowering effects and activity against osteoporosis and it is approved for the treatment and prevention of osteoporosis in postmenopausal women.

Mequitazine is the generic name of the compound 10-(Quinuclidin-3-ylmethyl)-10H-phenothiazine, having the following general formula (III)

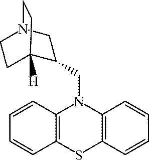

The compound is a histamine H₁ receptor antagonist and it is approved for use in the treatment of allergic conjunctivitis, allergic rhinitis, pruritus and urticaria.

N-tert-Butylisoquine is the generic name of the compound 2-[(tert-butylamino)methyl]-5-[(7-chloroquinolin-4-yl)amino]phenol, an antimalarial drug, having the following general formula (IV):

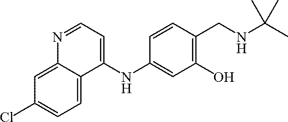

Isofloxythepin is the generic name of the compound 2-[4-(9-fluoro-3-propan-2-yl-5,6-dihydrobenzo[b][1]benzothiepin-5-yl)piperazin-1-yl]ethanol, having the following general formula (V):

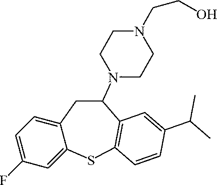

The compound is an antipsychotic agent.

Succinobucol is the generic name of the compound Succinic acid 2,6-di-tert-butyl-4-[1-(3,5-di-tert-butyl-4-hydroxyphenylsulfanyl)-1-methylethylsulfanyl]phenyl monoester (VI):

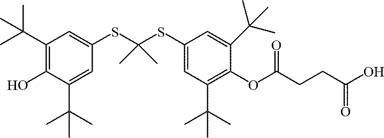 [SMILES: CC(C)(Sc1cc(C(C)(C)C)c(O)c(C(C)(C)C)c1)Sc1cc(C(C)(C)C)c(OC(=O)CCC(=O)O)c(C(C)(C)C)c1]

The compound is an antioxidant that was under clinical development for the treatment of atherosclerosis of the blood vessels of the heart, or coronary artery disease.

Hypericin is the generic name of the anthraquinone derivative 1,3,4,6,8,13-Hexahydroxy-10,11-dimethylphenanthro[1,10,9,8-opqra]perylene-7,14-dione, having general formula (VII):

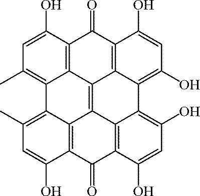

The compound is under development for the treatment cutaneous T-cell lymphoma. These compounds are therefore useful for the treatment of subjects infected by SARS-Cov-2 patients or for the prevention and/or treatment of contamination of a surface, including a body surface, or product with SARS-Cov-2.

Accordingly, a first object of the invention is a compound selected from Proflavine, Raloxifen, Mequitazine, N-tert-Butylisoquine, Isofloxythepin, Succinobucol and Hypericin or salts thereof, for use in the treatment of a SARS-CoV-2 infection in a subject.

A second object of the invention is a pharmaceutical composition comprising i) at least one compound according to the first object of the invention, and ii) at least one inert pharmaceutically acceptable excipient, for use in the treatment of a SARS-CoV-2 infection in a subject.

A third object of the invention is a method of treatment in the treatment of a SARS-CoV-2 infection in a subject, comprising administering to such subject a compound according to the first object of the invention.

A fourth object of the invention is the use of Proflavine, preferably Proflavine hemisulfate, for the prevention and/or treatment of contamination of a surface with SARS-CoV-2.

### DETAILED DESCRIPTION OF THE INVENTION

A first object of the present invention is a compound selected from Proflavine, Raloxifen, Mequitazine, N-tert-Butylisoquine, Isofloxythepin, Succinobucol, Hypericin, and salts thereof for use in the treatment of a SARS-CoV-2 infection in a subject.

A further object of the present invention is a compound selected from Proflavine, Raloxifen, Mequitazine, N-tert-Butylisoquine, Isofloxythepin, Succinobucol, Hypericin, and salts thereof for use in the treatment of a subject infected with SARS-CoV-2. According to the present invention, the term “subject” refers to a human or animal being. Preferably, said subject is a human being.

According to the present invention, the subject having a SARS-CoV-2 infection to be treated may symptomatic, paucisymtpomatic or asymptomatic.

Preferably, said subject has COVID-19. Accordingly, a further object of the present invention is a compound selected from Proflavine, Raloxifen, Mequitazine, N-tert-Butylisoquine, Isofloxythepin, Succinobucol, Hypericin, and salts thereof for use in the treatment of COVID-19 in a subject.

A particularly preferred compound for use according to the first object of the invention is Raloxifen or a salt thereof, preferably Raloxifen hydrochloride.

Another particularly preferred compound for use according to the first object of the invention is Hyperacin or a salt thereof.

A particularly preferred salt of Proflavine according to the invention is Proflavine hemisulfate or dihydrochloride.

As will be described in details in the experimental section, the present inventors have found that, in addition to their known activity, the above diverse compounds share the ability to bind and inhibit the activity of essential pathways of SARS-Cov-2 and thus to inhibit replication of the virus.

The selected compounds have the advantage that they have already been approved for clinical use or tested as safe in humans, in some cases are marketed drugs, and can therefore provide a rapid therapeutic approach to treatment of humans who are infected with SARS-CoV-2, preferably to the treatment of COVID-19 patients.

The compound for use according to the first object of the invention is administered in form of a pharmaceutical composition where the compound is admixed with one or more pharmaceutically acceptable excipients.

The compound according to the first object of the invention may be administered alone. Alternatively, combinations of compounds according to the first object of the invention may be used. According to the latter, a combination of two compounds selected from Proflavine, Raloxifen, Mequitazine, N-tert-Butylisoquine, Isofloxythepin, Succinobucol, Hypericin, and salts thereof is used in combined therapy. The two compounds can be administered simoutaneously or subsequently.

As preferred embodiments, the two compounds are Raloxifen and Proflavine, Raloxifen and Mequitazine, Raloxifen and N-tert-Butylisoquine, Raloxine and Isofloxythepin, Raloxifen and Succinobucol or Raloxifen and Hypericin or salts thereof.

As preferred embodiments, the two compounds are Hyperacin and Raloxifen, Hyperacin and Proflavine, Hyperacin and Mequitazine, Hyperacin and N-tert-Butylisoquine, Hyperacin and Isofloxythepin, Hyperacin and Succinobucol or salts thereof.

As preferred embodiments, the two compounds are Proflavine and Mequitazine, Proflavine and N-tert-Butylisoquine, Proflavine and Isofloxythepin, Proflavine and Succinobucol, Proflavine and Hypericin or salts thereof.

Thus, a second object of the invention is a pharmaceutical composition comprising at least one compound according to the first object of the invention and at least one inert pharmaceutically acceptable excipient, for use in the treatment of a subject infected with SARS-CoV-2, as described above.

According to a preferred emobodiment, the pharmaceutical composition comprises one compound according to the first object of the invention.

According to an alternative embodiment, the pharmaceutical composition comprises two compounds according to the first object of the invention selected from Proflavine, Raloxifen, Mequitazine, N-tert-Butylisoquine, Isofloxythepin, Succinobucol, Hypericin and salts thereof.

As preferred embodiment, the two compounds are Raloxifen and one compound selected from Proflavine, Mequitazine, N-tert-Butylisoquine, Isofloxythepin, Succinobucol, Hypericin and salts thereof.

As another preferred embodiment, the two compounds are Hyperacin and one compound selected from Raloxifen, Proflavine, Mequitazine, N-tert-Butylisoquine, Isofloxythepin, Succinobucol, and salts thereof.

As another preferred embodiment, the two compounds are Proflavine and one compound selected from Raloxifen, Mequitazine, N-tert-Butylisoquine, Isofloxythepin, Succinobucol, Hypericin and salts thereof.

The administration of the compound or pharmaceutical composition of the present invention may be systemic or topical.

Preferably, when the compound for use according to the first object of the invention is Proflavine or a salt thereof, this is for use in the treatment of a local SARS-CoV-2 infection in said subject by topical administration.

The administration of the compound or pharmaceutical composition of the present invention to a patient is in accord with known methods in the art and may be oral administration, comprising from one to several oral administrations per day, parenteral administration (including intravenous, intraperitoneal, intracerebral, intrathecal, intracranial, intramuscular, intraarticular, intrasynovial, intrasternal, intraocular, intraarterial, subcutaneous, intracutaneous or intralesional injection or infusion techniques), topical, buccal and suppository administration.

The route of administration of the compound or pharmaceutical composition of the present invention depends on the specific compound used or contained in the pharmaceutical composition and on the site of infection of SARS-Cov2 and it is in accordance with known methods for administration of the compound.

The pharmaceutical composition of the present invention may be formulated into oral, inhalatory or injectable dosage forms such as, for example tablets, capsules, powders, solutions, suspensions, and emulsions.

Preferably, the pharmaceutically acceptable excipient in the pharmaceutical composition includes any and all solvents, diluents, or other vehicle, dispersion or suspension aids, surface active agents, isotonic agents, thickening or emulsifying agents, preservatives, solid binders, lubricants and the like, as suited to the particular dosage form desired.

Some examples of materials which can serve as pharmaceutically acceptable excipient include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; cocoa butter and suppository waxes; oils such as peanut oil, cottonseed oi, safflower oil, sesame oil, olive oil, corn oil and soybean oil; glycols, such a propylene glycol; esters such as ethyl oleate and ethyl laurate; agar; buffering agents; alginic acid; pyrogen-free water; salts for regulating osmotic pressure; sterilized water; ethyl alcohol; preservatives, stabilizers, surfactants, emulsifiers, sweeteners, colorants, flavourings and the like.

The pharmaceutical composition of the present invention may be suitably formulated using appropriate methods known in the art, for example the methods disclosed in Remington’s Pharmaceutical Science (recent edition), Mack Publishing Company, Easton Pa.

In one preferred embodiment, the invention provides for oral administration of the compound or the pharmaceutical composition of the invention

In such embodiment, the pharmaceutical composition is preferably in form of a tablet or capsule.

According to an alternative preferred embodiment, the compound or pharmaceutical composition of the invention is administered parenterally, preferably by intravenous administration or continuous infusion. This route of administration is particularly suitable for intubated or critically ill COVID-19 patients.

In yet an alternative preferred embodiment, the invention provides for direct administration of the compound or pharmaceutical composition of the present invention to the respiratory tract. The direct administration to the respiratory tract may be the sole route of administration of the compound or composition or may be combined with administration of the compound or composition via other systemic or topical routes.

For direct administration of the compound or composition of the invention to the respiratory tract, a wide variety of devices are known in the art. In one aspect of the present invention, the compound or pharmaceutical composition of the present invention is administered in aerosolized or inhaled form.

The pharmaceutical composition of the present invention for direct administration to the respiratory tract as described above, can be in form of an aerosol formulation, as a dry powder or as a solution or suspension in a suitable diluent.

The dosage regimen of the compound according to the invention is that usually employed for other indications of the specific compound.

The dose and frequency of administration of the compound for use in the treatment according to the invention will vary with the active ingredient(s) being employed, the pharmaceutically-acceptable excipient(s)/carrier(s) utilized, the severity of the condition being treated, the patient’s age, body weight, general health status and sex, the chosen route of administration and dosage form, the number of administrations per day, the duration of the treatment, the nature of concurrent therapyand like factors within the knowledge and expertise of the attending physician. A person skilled in the art can determine the optimum posology in easily and routinely manner on the basis of the known pharmokinetic properties and dosage regime of the compound.

For example when the compound is Raloxifen or a salt thereof, preferably Raloxifen hydrochloride, this is preferably administered to the subject to the treated orally at a dosage of 120 mg per day, in one or two administrations. The total daily dosage may therefore be administered in a single 120 mg dose or divided in two daily 60 mg doses. As the skilled artisan will appreciate, lower or higher doses than those recited above may be required depending on specific istances.

A third object of the invention is a method of treating a SARS-CoV-2 infection in a subject, as above described, comprising administering to a patient affected thereby a compound selected from Proflavine, Raloxifen, Mequitazine, N-tert-Butylisoquine, Isofloxythepin, Succinobucol, Hypericin and salts thereof.

Due to their antiviral activity, the presently claimed compounds can also be used as antimicrobial agents to decontaminate surfaces from the virus. In particular, Proflavine, preferably in form of dichloride or hemisulfate, is particular useful for use as an antiviral agent to eliminate contamination of the virus from a surface, preferably a body surface.

Accordingly, a fourth object of the invention is the use of Proflavine or a salt thereof, preferably Proflavine dichloride or hemisulfate, as an antimicrobial agent for the prevention and/or treatment of contamination of a surface by SARS-CoV-2.

Preferably, said surface is a body surface.

The invention will be further described in the following examples, which do not limit the scope of the invention described in the claims.

### EXPERIMENTAL PART

### 1. Computer Associated Drug Design

The EXSCALATE platform, the most powerful computing resources currently based in Europe to empower smart in-silico drug design (co-funded by the H2020-FET-HPC ANTAREX project), was exploited to perform molecular dynamics (MD) and docking simulations, with the final aim to select and make available molecules active against the SARS-CoV-2 proteins.

The crystal structures of the main functional units of SARS-CoV-2 proteome were obtained from the Protein Data Bank;

Table 1 below reports the list of the proteins analysed, with the corresponding PDB code. Homology models of the proteins for which the crystal structure is not available were generated and used. In particular, the SARS-CoV-2 proteins most interesting as target to identify antiviral drugs, were selected.

TABLE 1

| | |
|---|---|
| Proteins | PDB code |
| 3CL protease | 6LU7 |
| N-protein | 6VYO |
| NSP3 | 6W02 |
| NSP6 | De novo model |
| NSP9 | 6W4B |
| NSP12 | 7BV2 |
| NSP13 | Homology Model |
| NSP14 | Homology Model |
| NSP15 | 6W01 |
| NSP16 | 6W4H |
| PL protease | 6W9C |
| Spike-ACE2 | 6M0J |

Molecular dynamics simulations on SARS-CoV-2 proteins were performed to explore the conformational space of the active sites and to select several protein conformations particularly suitable for docking simulations.

In the first step, MD simulations were carried out on the proteins structures, prepared ad hoc in order to optimize the 3D structures from a chemical and conformational point of view. The structures were firstly subjected to a cycle of energy minimization by steepest descent methods to eliminate all initial steric clashes and obtain a pre-equilibrated model to start from. Then a 100 ps restrained MD simulation (typically 1000 KJ/mole force constant) was performed on the solvent atoms to equilibrate water molecules keeping the solute restrained. Finally, a production run was performed to generate a 1 microsecond trajectory with a total of 20.000 collected. Post HPC-run analysis of the results was performed.

In a second step a High Performance Computing (HPC) simulation was conducted to virtual screen the Safe in Man (SIM) library, containing commercialized and under development drugs, already proved safe in man (> 10.000 drugs), against the proteins of SARS-CoV-2, on both the crystal structures and the most 100 relevant conformations extracted from MD runs.

The SIM library is a Dompe dataset containing about 10.000 pharmaceutical compounds, built merging the list of drugs launched or under active development in several clinical phases with molecules in early phases (biological testing and preclinical) matching the query “CoV Inhibitors” as mechanism of action. Both lists derived from an Integrity database search (https://clarivate.com/cortellis/solutions/pre-clinical-intelligence-analytics/). All the compounds have been classified based on the mechanism of action of the class (antibacterial, antiviral, antiparasitic, antifungal, etc...) and on merging information reported in the Integrity database with those included in the drug repositioning database “RepoDB”. Also drug information provided by DrugCentral, related to pharmaceutical products, drug mode of action, indications and pharmacologic action, was integrated in Dompe drug virtual library, as well as information on drugs and drug targets reported in the DrugBank database.

Ligand were prepared with Schrödinger’s LigPrep tool. This process generated multiple states for stereoisomers, tautomers, ring conformations (1 stable ring conformer by default) and protonation states. In particular, another Schrödinger package, Epik, was used to assign tautomers and protonation states that would be dominant at a selected pH range (pH=7±1). Ambiguous chiral centers were enumerated, allowing a maximum of 32 isomers to be produced from each input structure. Then, an energy minimization was performed with the OPLS3 force.

The simulation was performed using LiGen™ (Ligand Generator), the de novo structure based virtual screening software, designed and developed to run on HPC architectures, which represent the most relevant tool of the EXSCALATE platform. LiGenTM is formed by a set of tools that can be combined in a user-defined manner to generate project centric workflows. In particular, LiGenDock is a docking module using LiGenScore to compute the scoring function and the LiGenPass and LiGenPocket modules to obtain the 3D structure of the binding site. Both the docking algorithm implemented in LiGen, the pharmacophoric docking (LiGenPh4) and the geometrical docking (LiGenGeodock), as well as the different scoring functions calculated, were used in this study to explore different protocols of Virtual Screening (VS) and select the best one in terms of performance.

Usually, the performance of a VS protocol is assessed by evaluating its capacity to recognize the active molecules among a large number of inactive decoys, where usually the active molecules represent the 1% of the total number of compounds. The performances of the tested VS strategy was assessed by evaluating the capacity to correctly rank molecules which are endowed with antiviral activity, and in particular with a known effect against coronaviruses. Following this approach, about 130 molecules were labelled as active compounds.

The VS protocol performance was thus evaluated comparing the screening results of the SIM library on the crystal structure of 3C-like protease of SARS-CoV-2 and on the most 100 relevant conformations extracted from MD runs, by using different docking algorithms and different scoring function. The conditions showing the best capacity to recognize the active molecules were used to prioritize the total list of screened molecules and select the top scored compounds, according to the score value (Csopt), that predicts the binding affinity of the molecules in the protein binding site.

Virtual screening was performed on the main SARS-CoV-2 proteins to evaluate the potential poly-pharmacological effect of the compounds on the COVID-19 virus.

A total score, corresponding to the sum of the docking scores obtained for each protein, was also calculated for each molecule and used to prioritize the most interesting molecules.

The molecules Proflavine, Raloxifen, Mequitazine, N-tert-Butylisoquine, Isofloxythepin, Succinobucol and Hypericin were selected among the best scored molecules in terms of total_score, meaning polipharmacological profile, for further validation of the inhibitory activity in in vitro experiments.

Table 2 and 3 report the results obtained for the selected compounds for each of the proteins, while Table 4 reports the total score obtained for each compound.

TABLE 2

| Compound | 3CL | PLPro | NSP3 | NSP6 | NSP9 | NSP12 AlloPalm | NSP12 AlloThumb | NSP12 Orto |
|---|---|---|---|---|---|---|---|---|
| Hypericin | 7.16 | 6.37 | 7.66 | 6.72 | 6.07 | 6.45 | 6.13 | 6.02 |
| Raloxifen hydrochloride | 6.53 | 6.35 | 7.74 | 6.62 | 5.96 | 6.45 | 5.83 | 5.42 |
| N-tert-Butylisoquine | 6.43 | 6.04 | 6.69 | 6.24 | 5.71 | 5.94 | 5.74 | 5.35 |
| Isofloxythepin | 6.39 | 5.91 | 6.64 | 6.26 | 5.16 | 5.71 | 5.49 | 5.29 |
| Succinobucol | 7.45 | 6.78 | 0 | 6.68 | 0 | 6.4 | 6.07 | 6.04 |
| Mequitazine | 5.84 | 5.58 | 6.17 | 5.86 | 4.94 | 5.48 | 5.29 | 5.07 |
| Proflavine | 5.06 | 5.21 | 5.63 | 5.03 | 4.5 | 4.89 | 5.09 | 4.65 |

TABLE 3

| Compound | NSP13orto | NSP13allo | NSP14 | NSP15 | NSP16 | SPIKE | N-prot |
|---|---|---|---|---|---|---|---|
| Hypericin | 6.16 | 6.26 | 8.25 | 6.72 | 6.94 | 6.82 | 6.6 |
| Raloxifen hydrochloride | 5.79 | 5.98 | 7.62 | 6.51 | 6.88 | 6.51 | 6.46 |
| N-tert-Butylisoquine | 5.48 | 5.79 | 6.77 | 6.2 | 6.54 | 6.18 | 6.2 |
| Isofloxythepin | 5.31 | 5.65 | 6.71 | 6.17 | 6.33 | 6.26 | 5.93 |
| Succinobucol | 6.38 | 6.63 | 8.08 | 6.55 | 7.44 | 6.91 | 6.88 |
| Mequitazine | 5.19 | 5.27 | 6.35 | 5.84 | 5.97 | 5.94 | 5.4 |
| Proflavine | 4.75 | 4.88 | 5.71 | 5.24 | 5.16 | 4.87 | 5.12 |

TABLE 4

| Compound | Total_score |
|---|---|
| Hypericin | 100.35 |
| Raloxifen hydrochloride | 96.64 |
| N-tert-Butylisoquine | 91.29 |
| Isofloxythepin | 89.19 |
| Succinobucol | 88.3 |
| Mequitazine | 84.19 |
| Proflavine | 75.78 |

### 2. In Vitro Screening of Activity Against SARS-CoV-2

The results obtained by this screening have been confirmed in in vitro experiments that have validated their antiviral activity.

The compounds selected by the CADD analysis were tested in a SARS-CoV 2 VeroE6-EGFP HTS antiviral Assay. This assay is based on viral infection of VeroE6 cells followed by visual monitororing of cytopathic effects.

In details, the EGFP-fluorescent-reporter constitutively expressing cells line VeroE6-EGFP was received from JNJ whereas the SARS-CoV-2 was received from a Belgian strain (BetaCov/Belgium/GHB-03021/2020).

The test compounds were dissolved at 10 mM in dimethylsulphoxide (DMSO) and then diluted in cell culture medium to a final DMSO concentration below 0.5% and mixed with 30 CCID50 of SARS-CoV and 2000 VeroE6-EGFP cells per well in 384-well plates. As a control, wells containg 30 CCID50 of SARS-CoV and 2000 VeroE6-EGFP cells per well were also set up.

After incubation at 37° C. for 5 days the EGFP signal of each well was recorded using an ArrayScan, which is a LED-based high-content imager.

Standard whole-well fluorescence plate readout was performed (self-optimizing protocol, ~6 min per 384-well plate, 4 reads/well) and the fluorescence total intensity of the test compounds wells and of the control wells was measured.

High-content imaging readout was also performed using a 5x objective, allowing to capture almost the entire well of a 384-well plate at once (auto-focus on each well, no binning, 1 channel). Two values were obtained for each compounds from high content imaging, shown in Table 5:

% Confluence: this is the percentage increase in confluence in the test compound wells compared to the control wells, based on readout “SpotTotalAreaCh2” which is the total surface of the field of view that is green. Since the GFP marker is located in both the cell cytoplasm and in the nucleus, it allows to calculate the surface of the well that is (still) covered by cells (a large value means that there are still a lot of cells on the microtiter plate bottom surface, a small value means that most of the fluorescence i.e. the cells are gone).

% Inhibition: this is the percentage increase in the number of cells in the test compound wells compared to the control wells, based on “ValidObjectCount” which is the total count of the number of nuclei. The nuclei are brighter green than the cytoplasm, which allows to count the number of cells, providing that the cell density is not too high.

TABLE 5

| Compound | % Confluence | % Inhibition |
|---|---|---|
| Proflavine | 8.56 | 208.4 |
| Raloxifen hydrochloride | 45.26 | 70.75 |
| Mequitazine | 61.74 | 62.09 |
| N-tert-Butylisoquine | 77.86 | 44.99 |
| Isofloxythepin | 67.67 | 35.39 |
| Succinobucol | 18.73 | 37.53 |
| Hypericin | 20.85 | NA |

Furthermore, IC50 of each compound was obtained, from dose-response curves obtained testing the compounds antiviral effect at 8 different concentrations. This value indicates the half maximal concentration able to recover the cytopatic effect of infection and is a measure of the potency of a substance in inhibiting viral included cell death.

The results obtained for each of the compounds tested are shown in Table 6 below.

TABLE 6

| Compound | IC50 (µM) |
|---|---|
| Proflavine | 27.46 |
| Raloxifen hydrochloride | 9.78 |
| Mequitazine | 5.99 |
| N-tert-Butylisoquine | 3.43 |
| Isofloxythepin | 106.22 |
| Succinobucol | 17.21 |
| Hypericin | 0.25 |

As can be see from the results, all compounds are able to inhibit virus replication and consequent cytopathic effects of the virus compared to the control.

Furthermore the ability of inhibiting the SARS-CoV-2 main protease and the Papain-like Protease Protein activities on a FRET based assay was evaluated.

The detection of enzymatic activity of the SARS-CoV-2 3CL-Pro was performed under conditions similar to those reported by Zhang et al (PMID: 32198291) and adjusted as follow. Enzymatic activity was measured by a Förster resonance energy transfer (FRET), using the dual-labelled substrate, DABCYL-KTSAVLQ↓SGFRKM-EDANS (Bachem #4045664) containing a protease specific cleavage site after the GIn. In the intact peptide, EDANS fluorescence is quenched by the DABCYL group. Following enzymatic cleavage, generation of the fluorescent product was monitored (Ex/Em= 340/460 nm), (EnVision, Perkin Elmer).

A biochemical assay for detection of SARS-CoV-2 PLpro enzymatic activity was also developed in accordance with recent publications by Shin et al., 2020 (https://doi.org/10.1038/s41586-020-2601-5). Here we use a commercial source of protein (BPS Bioscience #100735) and a fluorescently labeled ISG15 as a substrate (BostonBiochem ISG15/UCRP AMC, #UL-553). The assay was performed in a buffer containing 50 mM Tris (pH 7.5) and 150 mM NaCl, using 100 nM of SARS-CoV-2 3CLpro and 2.5 µM FRET-substrate.

For both assays we performed a primary screen in which test compounds (stock at 10 mM in 100% DMSO), positive (zinc pyrithione (medchemexpress, #HY-B0572) 10 mM in 100% DMSO) and negative (100% DMSO) controls, were transferred to 384-well assay microplates by acoustic dispensing (Echo, Labcyte). 5 µl of SARS-CoV-2 3CL-Pro stock (120 nM) in assay buffer were added to compound plates and incubated for 60 min at 37° C. This pre-incubation step facilitated the identification of slowly binding putative cysteine-reactive inhibitors. After addition of 5 µl substrate (30 µM in assay buffer), the final concentrations were: 15 µM substrate; 60 nM SARS-CoV-2 proteases; and 0.2% DMSO in a total volume of 10 µL/well. The fluorescence signal was then measured at 15 min and inhibition (%) calculated relative to controls (Envision, PerkinElmer). Results were normalized to the 100% (positive control) and 0% (negative control) inhibition. To flag possible optical interference effects, primary assay plates were also read 60 min after substrate addition, when the reaction was complete. In this experimental set, some compounds used as positive controls slow down or lose their inhibition activity in the presence of DTT. So to, account for any DTT dependent effects, primary screening was performed without DTT in the assay buffer.

Primary screening to assess proteases inhibition was performed at a unique test compound concentration attested at 20 µM. The resulting compounds were profiled in dose response achieved with serial dilution at 8 different concentration points following a log dilution (starting concentration 0.033 µM) in absence of DTT.

To select the compound we evaluated the following parameters:

Inhibition (%): This parameter is strictly linked to fluorescence signal registered during the assay. Given that positive and negative control fluorescence is considered 100% and 0%, respectively; the fluorescence registered in compounds wells is reported to this scale. Interestingly, all the selected compounds have an inhibition higher than 80%.

For three of the compounds selected the activity on SARS-CoV-2 3CL-Pro and PLPro-Pro was confirmed. The results obtained for these compounds are reported in Table 7.

TABLE 7

| Compound | 3CL-Pro Inhibition [%] | PL_Pro Inhibition [%] |
|---|---|---|
| Raloxifen hydrochloride | 20.00 | 60.00 |
| Proflavine | 89.86 | |
| Hypericin | 45.67 | 40.11 |

1. A method for treating a SARS-CoV-2 infection in a subject, the method comprising administering a compound selected from the group consisting of Proflavine, Raloxifen, Mequitazine, N-tert-Butylisoquine, Isofloxythepin, Succinobucol, Hypericin, and salts thereof to the subject.

2. The method of claim 1, wherein said subject has COVID-19.

3. The method of claim 1, wherein said subject is symptomatic, paucisymtpomatic or asymptomatic.

4. The method of claim 3, wherein said compound is Raloxifen or a salt thereof.

5. The method of claim 4, wherein Raloxifen or said salt thereof is administered to said subject orally at a dosage of 120 mg per day, in one or two administrations.

6. The method of claim 1, where said compound is Proflavine or a salt thereof.

7. The method of claim 6, wherein said infection is a local infection and said Proflavine or a salt thereof is administered by topical administration.

8. A method for treating SARS-CoV-2 infection in a subject, the method comprising administering to the subject a pharmaceutical composition comprising:
i) at least one compound selected from the group consisting of Proflavine, Raloxifen, Mequitazine, N-tert-Butylisoquine, Isofloxythepin, Succinobucol, Hypericin, and salts thereof subject, and
ii) at least one inert pharmaceutically acceptable excipient.

9. The method of claim 8, wherein the pharmaceutical composition contains only one said compound.

10. The method of claim 8, containing a combination of two compounds selected from:
Raloxifen and one compound selected from Proflavine, Mequitazine, N-tert- Butylisoquine, Isofloxythepin, Succinobucol, Hypericin and salts thereof;
Hyperacin and one compound selected from Raloxifen, Proflavine, Mequitazine, N-tert-Butylisoquine, Isofloxythepin, Succinobucol, and salts thereof; or
Proflavine and one compound selected from Raloxifen, Mequitazine, N-tert- Butylisoquine, Isofloxythepin, Succinobucol, Hypericin and salts thereof.

11. The method of claim 1, wherein said compound is administered orally.

12. The method of claim 1, wherein said compound is administered parenterally.

13. A method for the prevention and/or treatment of a surface from SARS-CoV-2 contamination, the method comprising contacting the surface with Proflavine or a salt thereof.

14. The method of claim 13, wherein said surface is a body surface.

15. The method of claim 4, wherein the compound is Raloxifen hydrochloride.

16. The method of claim 6, wherein the compound is Proflavine dichloride or hemisulfate.

17. The method of claim 13, wherein the salt of Proflavine is Proflavine dichloride or hemisulfate.
